(19) United States
(12) Patent Application Publication
(10) Pub. No.: US 2017/0304231 A9
(43) Pub. Date: Oct. 26, 2017

(54) METHODS OF DIAGNOSIS AND TREATMENT

(71) Applicant: Theranostics Lab, Cleveland, OH (US)

(72) Inventors: Patrick Alton Gladding, Auckland (NZ); Mia Jullig, Waiheke Island (NZ); Silas Villas-Boas, Cleveland, OH (US); Seif El-Jack, Auckland (NZ)

(21) Appl. No.: 14/877,367

(22) Filed: Oct. 7, 2015

Related U.S. Application Data
(62) Division of application No. 13/837,078, filed on Mar. 15, 2013, now abandoned.
(60) Provisional application No. 61/615,118, filed on Mar. 23, 2012.

(51) Int. Cl. A61K 31/138; A61K 47/36; A61K 9/107

(52) U.S. Cl. CPC A61K 31/138; A61K 9/1075; A61K 47/36

(57) ABSTRACT

A method for diagnosing a cardiac ischemic event and a kit for detecting biomarkers used in the method. Also provided is a method for treating a PPAR-related disorder by administering a pharmaceutical composition containing one or more long chain fatty acids.

### CROSS-REFERENCE TO RELATED APPLICATIONS

This application is a divisional of U.S. patent application Ser. No. 13/837,078, filed Mar. 15, 2013, which claims the benefit of U.S. Application No. 61/615,118, filed Mar. 23, 2012, each of which are incorporated by reference in their entireties.

### FIELD

The present invention relates to methods for diagnosing a cardiac ischemic event and to the treatment of peroxisome proliferator-activated receptor (PPAR) related disorders in a subject.

### BACKGROUND

Cardiovascular disease places a substantial burden on healthcare resources given the high morbidity and significant mortality associated with the disease.

Cardiac ischemia occurs when blood flow does not meet demand to the heart muscle. The term includes a number of disorders including, for example, heart attack, myocardial infarction, and angina. Cardiac ischemia contributes significantly to hospitalization and mortality.

Misdiagnosis of cardiac ischemia can lead to the wrong treatment plan being implemented, increasing the risk and cost to the patient and to the public and private health systems.

Methods for diagnosing cardiac ischemia are known, but they may present with problems. For example, the method of diagnosis may be invasive, expensive, or slow.

### OBJECT

It is an object of the present invention to provide a method of diagnosis, a method of treatment, a composition, a medicament, a kit, and/or uses which overcomes or ameliorates at least one of the disadvantages of known methods, compositions, medicaments, kits, or uses or to at least provide the public with a useful choice.

### STATEMENT OF INVENTION

According to a first broad aspect of the invention, there is provided a method for diagnosing a cardiac ischemic event comprising at least the step of determining in a sample from a subject the level of one or more biological markers chosen from the group consisting of one or more long chain fatty acids and CD44.

In a second aspect of the invention, there is provided a method for diagnosing a cardiac ischemic event in a subject, the method comprising at least the steps of:
a) detecting the level of the one or more biological markers in one or more sample from the subject; and,
b) comparing the level of the one or more biological markers against one or more standard; wherein a difference in the level of the one or more biological markers in one or more sample compared to one or more standard is diagnostic of a cardiac ischemic event.

In one embodiment, the method is for diagnosing a recent cardiac ischemic event.

In one embodiment, the method comprises repeating the step a) of detecting the level of the one or more biological markers in one or more sample from the subject two or more times at discrete time points.

In one embodiment, the method comprises detecting the level of a combination of two or more of the biological markers. In other embodiments, the method comprises detecting the level of a combination of three or more, four or more, five or more, or six or more of the biological markers.

Preferably, the one or more long chain fatty acids are selected from oleic acid (C18:1), palmitic acid (C16:0), palmitoleic acid (C16:1), stearic acid (C18:0), pentadecanoic acid (C15:0) and myristic acid (C14:0).

In one embodiment, the method comprises detecting the level of oleic acid. In one embodiment, the method comprises detecting the level of myristic acid. In another embodiment, the method comprises detecting the level of both oleic acid and myristic acid.

In another embodiment, the method comprises detecting the level of CD44. In another embodiment, the method comprises detecting the level of both CD44 and oleic acid. In another embodiment, the method comprises detecting the level of both CD44 and myristic acid. In another embodiment, the method comprises detecting the level of CD44, oleic acid and myristic acid.

In one embodiment the method further includes the detection of one or more other biological marker. In one embodiment the one or more other biological marker is one or more metabolite. In one embodiment, the one or more metabolite is chosen from the group comprising tryptophan, glycine, lysine, isoleucine, leucine, hydroxybutyric acid, phenylalanine, valine, creatinine, threonine, aspartic acid, glutamic acid, pyroglutamic acid, alanine, cysteine, and lactic acid.

In another embodiment, the one or more metabolite may be chosen from one or more of glucose, lactate, glutamine, glycine, glycerol, phenylalanine, tyrosine, phosphoethanolamine, choline-containing compounds and triacylglycerols, total, esterified, and nonesterified fatty acids, fructose, myoinositol, pyruvate, lactate, oxalate, citrate, isocitrate, succinate, malate, valine, alanine, serine, glycine, cysteine, threonine, aspartate, tryptophan, tyrosine, 4-hydroxyproline2-hydroxybutyrate, 2-aminobutyrate, 2,3,4-trihydroxybutyrate3-hydroxybutyrate, creatinine and aminomalonate.

In other embodiments, the one or more other biological marker is chosen from aspartate aminotrasferase, lactate dehydrogenase, creatine kinase, hydroxybutyrate dehydrogenase, CK-MB (activity), CK-MB (mass), CK isoforms, myoglobin, carbonic anhydrase Ill, glycogen phosphorylase BB, Heart fatty acid binding protein, myosin light chains, pregnancy-associated plasma protein, choline, ischemia-modified albumin, unbound free fatty acids, placental growth factor, myeloperoxidase MMP-9, sCD40L and troponin I or T.

In addition, the method of the invention may also comprise the step of obtaining electrocardiography (ECG) and/or ultrasound data from the subject.

In one embodiment, the method of the invention may comprise obtaining data for one or more of myristic acid, oleic acid, and CD44 in combination with ECG data from the subject.

In another embodiment, the method of the invention may comprise obtaining data for one or more of myristic acid, oleic acid, and CD44 in combination with ultrasound data from the subject. In another embodiment, the method of the invention may comprise obtaining data for one or more of myristic acid, oleic acid, and CD44, in combination with both EGG and ultrasound data from the subject.

In one embodiment, the method comprises detecting the level of the one or more biological markers in each of two or more samples from the subject. In other embodiments, the method comprises detecting the level of one or more biological makers in each of three or more, or four or more samples taken from a subject. In one embodiment, the two or more samples are different. In another embodiment, the two or more samples are the same. In one embodiment, the two or more samples may be taken at different time points.

In one embodiment, the one or more sample can be selected from a breath sample, a blood sample, a urine sample or a plasma sample.

In one particular embodiment, the sample is a breath sample.

In one embodiment, the one or more biological markers is detected in both a blood and a breath sample.

In another embodiment, the one or more biological markers is detected in both a blood and a breath sample and electrocardiography (EGG) and/or ultrasound data is also obtained.

In another embodiment, the method comprises detecting the level of the one or more diagnostic marker in one sample and one or more different diagnostic markers in a different sample. Alternatively, the level of the same biological markers may be detected in each sample.

In one embodiment, the one or more standard is a level of the one or more biological markers associated with the absence of a cardiac ischemic event and a higher or increased level of the one or more biological markers in the sample compared to the one or more standard is diagnostic of a cardiac ischemic event.

Preferably, there is at least an approximately 1.5 fold increase or decrease in the level of one or more biological markers, more preferably at least an approximately 2 fold increase or decrease, compared to the one or more standard. In another embodiment, there is at least an approximately 5 fold increase or decrease in the level of the one or more biological markers.

In one embodiment, the one or more long chain fatty acids are detected using any one of mass spectrometry (GC-MS, LC-MS, MSMS), electrochemistry, or by the use of chemoresistive nanopolymers. In another embodiment, the one or more long chain fatty acids are detected using selected ion flow tube mass spectrometry (SIFT-MS).

In one embodiment, CD44 is detected using mass spectrometry or immunological techniques such as immunoassays including but not limited to enzyme linked immunosorbent assay (ELISA) (sandwich ELISA, double sandwich ELISA, direct ELISA, microparticle ELISA), radioimmunoassay (RIA), immunoprecipitation, western blotting, immunohistochemical staining, or agglutination assay.

In one embodiment, the methods of the invention may combine the use of two or more detecting techniques. In one embodiment, two or more techniques are used to analyze the same biological marker and/or the same sample. Where more than one biological marker is to be detected or more than one sample is to be analyzed, one or a combination of detection techniques may be used. The samples may be analyzed simultaneously or sequentially in any order.

In one embodiment, breath and blood samples are analyzed simultaneously.

In one embodiment, the method further comprises the step of treating a subject for cardiac ischemia where a difference in the level of the one or more biological markers in a sample compared to one or more standard is diagnostic of a cardiac ischemic event.

In another embodiment, the method further comprises the step of deciding not to treat a subject for cardiac ischemia where a difference in the level of the one or more biological markers in a sample compared to one or more standard is not diagnostic of a cardiac ischemic event.

In another embodiment, the method further comprises the step of measuring levels of one or more biological marker during or after treatment for cardiac ischemia to determine the effectiveness of the treatment.

In one embodiment, the method comprises the step of measuring the level of one or more biological markers at one or a combination of two or more distinct time points during or after treatment to determine the effectiveness of the treatment.

In one embodiment, the time points are chosen from the time of treatment, 20 minutes after treatment, 1 hour from treatment, 6 hours from treatment, and 8 hours from treatment. In another embodiment the time points are chosen from at least 12 hours, at least 18 hours, and at least 24 hours after treatment. In another embodiment, the time points are chosen from one to several weeks or one to several months after treatment. In one embodiment, the time points are chosen from 3 months and 6 months after treatment. Reference to the “time of treatment” is intended to include the first or any subsequent treatments.

In another aspect there is provided a system for diagnosis in relation to cardiac ischemia in a subject, said system comprising a processor coupled to a memory having one or more reference standards, the processor arranged, in response to receiving subject information including information on the level of one or more biological markers chosen from long chain fatty acids and CD44 for said subject, to compare the received subject information with the one or more reference standards in order to generate a diagnosis relating to cardiac ischemic in the subject, and communicating said diagnosis.

In an embodiment the fatty acids comprise one or more of: myristic acid, oleic acid.

In an embodiment the subject information additionally includes one or more of EGG data, ultrasound data, the level of one or more additional biomarkers, clinical observations and/or medical history.

The one or more reference standard may include a level of one or more biological marker comprising one or more long chain fatty acid and CD44 associated with cardiac ischemia, or the level of the one or more biological markers which indicate the absence of a cardiac ischemic event.

In addition, the memory may include one or more of standard ECG data and/or ultrasound data, standard information on the level of one or more additional biomarkers associated with cardiac ischemic or the absence of a cardiac ischemic event, historical clinical observations, medical history, genomic profile, family history, previous test results and other medically relevant data. This data may also be used by the processor in order to generate the diagnosis.

In an embodiment communicating the diagnosis comprises one or more of: displaying the diagnosis on a display device, transmitting data indicative of the diagnosis to a remote device such as a cellular phone, activating a visual and/or audible alarm.

In an embodiment receiving the subject information comprises one of more of: receiving data entered via a user keypad or other user interface, receiving data storage media containing said analysis, receiving the analysis from a remote device using a communications link.

In an embodiment the processor is further arranged to generate and communicate updated diagnoses in response to receiving updated subject information for said subject. The updated diagnoses may indicate one or more cardiac ischemic events. The updated diagnoses may indicate subject response to treatment by monitoring rising or falling levels of the biological markers over time. For example a warning may be communicated when levels rise over a threshold and/or over a predetermined gradient.

In another aspect there is provided a computerized method for diagnosing in relation to cardiac ischemia in a subject, said method comprising: receiving subject information including information on the level of one or more biological markers chosen from long chain fatty acids and CD44 for said subject; comparing the received subject information with reference standards in order to generate a diagnosis relating to cardiac ischemia in the subject; communicating said diagnosis.

In another aspect there is provided a computer program stored on a non-transitory data storage medium, the computer program when executed on a computer arranged to perform method for diagnosing in relation to cardiac ischemia in a subject, said method comprising: receiving subject information including information on the level of one or more biological markers chosen from long chain fatty acids and CD44 for said subject; comparing the received subject information with reference standards in order to generate a diagnosis relating to cardiac ischemia in the subject; communicating said diagnosis.

Although the use of computerized generation of a diagnosis has only been discussed with reference to the above embodiments, this can readily be applied to any of the diagnostic teachings within this specification.

In a third aspect of the invention, there is provided a pharmaceutical composition comprising one or more long chain fatty acids or salts thereof optionally in combination with one or more pharmaceutically acceptable diluents, carriers and/or excipients.

Preferably, the one or more long chain fatty acids are selected from oleic acid, palmitic acid, palmitoleic acid, stearic acid, pentadecanoic acid, and myristic acid.

Preferably, the long chain fatty acid is selected from oleic acid and myristic acid.

In one embodiment, the pharmaceutical composition comprises oleic acid. In one embodiment, the pharmaceutical composition comprises myristic acid. In another embodiment, the pharmaceutical composition comprises oleic acid and myristic acid.

In a fourth aspect of the invention, there is provided a method for treating a PPAR-related disorder. In one embodiment the disorder is one resulting from aberrant cellular differentiation, cell growth, metabolism, inflammation or tumorogenesis, the method comprising at least the step of administering a pharmaceutical composition of the invention to a subject.

Preferably, the disorder is selected from cardiac ischemia, diabetes and malaria.

In one embodiment, the pharmaceutical composition comprises one or more long chain fatty acid.

In one embodiment, the pharmaceutical composition comprises oleic acid. In one embodiment, the pharmaceutical composition comprises myristic acid. In another embodiment, the pharmaceutical composition comprises oleic acid and myristic acid.

Preferably, the pharmaceutical composition is administered orally, by inhalation, or by injection such as cutaneous, subcutaneous, or intravenous injection.

In a fifth aspect of the invention, there is provided a micelle, wherein the micelle comprises a cell targeting molecule. In one embodiment, the micelle contains one or more pharmaceutical compound or composition.

In one embodiment, the pharmaceutical composition is a pharmaceutical composition of the invention. In another embodiment, the pharmaceutical compound or composition comprises dexamethasone.

In one embodiment the cell targeting molecule is a ligand that is capable of binding to a target receptor on a cell.

Preferably, the ligand is hyaluronic acid (HLA).

Preferably, the target receptor is CD44.

In one embodiment, the micelle comprises imaging agents to enable visualization of the delivery of the pharmaceutical composition of the invention.

In one embodiment, the imaging agents are fluorescent imaging agents.

In one embodiment, the imaging agents are radio-isotopes.

In one embodiment, the imaging agents are quantum dots. In one embodiment, the quantum dots comprise silicon.

In a sixth aspect of the invention, there is provided a use of a pharmaceutical composition of the invention in the manufacture of a medicament for the treatment of a PPAR-related disorder.

In one embodiment, the disorder is cardiac ischemia. In one embodiment, the disorder is diabetes mellitus. In one embodiment, the disorder is malaria.

In one embodiment, the pharmaceutical composition comprises one or more long chain fatty acid.

In one embodiment, the pharmaceutical composition comprises oleic acid. In one embodiment, the pharmaceutical composition comprises myristic acid. In another embodiment, the pharmaceutical composition comprises oleic acid and myristic acid.

In a seventh aspect of the invention, there is provided a kit for use in a diagnostic method of the invention, the kit comprising at least one or more reagents suitable for detection of one or more biological markers chosen from the group consisting one or more long chain fatty acids and CD44 as herein before described.

In another aspect, the invention provides the use of a ligand of CD44 to target an agent for delivery to a cardiac cell, circulating peripheral blood cell, endothelial cell, multipotent haemopoietic stem cell or rare circulating cell. It also provides constructs and compositions comprising an agent to be delivered to a cardiac cell, circulating peripheral blood cell, endothelial cell, multipotent haemopoietic stem cell or rare circulating cell and a ligand for CD44.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE DRAWINGS

FIG. 1 is a schematic diagram of a system for diagnosis in relation to cardiac ischemia in a subject. The system (100) comprises a processor (105) coupled to a memory (110) having one or more reference standards (115a), a computer program (105b), and one or more of standard ECG data and/or ultrasound data (115c). A user interface (130) coupled to the diagnostic system (100), and comprises a display (120) and a keyboard and/or mouse combination (125) for entering data into the diagnostic system (100). There may also be provided a remote device (135) such as a mobile phone for receiving information from the diagnostic system (100).

FIG. 2 is a flow diagram illustrating a computerized method for diagnosing in relation to cardiac ischemia in a subject, and which may be implemented in the system of FIG. 1.

FIGS. 3A and 3B are flow diagrams illustrating algorithms for generating a diagnosis.

### DETAILED DESCRIPTION OF PREFERRED EMBODIMENTS

The following is a description of the present invention, including preferred embodiments thereof, given in general terms. The invention is further elucidated from the disclosure given under the heading “Examples” which provides experimental data supporting the invention and specific examples thereof.

The inventor(s) have surprisingly identified that the long chain fatty acids, myristic acid and oleic acid, are altered in the blood of patients following cardiac ischemic events. They have also surprisingly identified that CD44 are altered in the blood of patients following cardiac ischemic events. The inventors believe the biological markers identified, and their relative levels, can be used as markers in the diagnosis of cardiac ischemic events. Of particular advantage is the observation that differences in levels of these biological markers may be detected within 20 minutes of symptom onset. The inventor(s) believe this may allow for earlier diagnosis and medical intervention in the case of cardiac ischemic events. Having studied the results obtained, the inventors believe long chain fatty acids, other than myristic and oleic acid, may also be of diagnostic use for cardiac ischemic events. They also contemplate diagnosis being achieved within 5 minutes of symptom onset.

The inventors have also identified that a ligand for CD44 can be used to target an agent for delivery to cardiac cells, circulating peripheral blood cells, endothelial cells, multipotent haemopoietic stem cells, or rare circulating cells.

In addition, preliminary studies by the inventors indicate that myristic acid, oleic acid, and other long chain fatty acids may be used for the treatment of cardiac ischemia and a number of other disorders.

Accordingly, described herein is a method for the diagnosis of a cardiac ischemic event comprising at least the step of observing or detecting the level of one or more biological marker chosen from the group consisting of one or more long chain fatty acid and CD44 in a sample from a subject. As used herein “detecting the level” should be taken broadly and should not be taken to imply that the marker must be present in a sample. It should be taken to include reference to detecting that a biological marker is not present in a sample.

The inventors contemplate that the level of one or more fragments of the one or more biological markers may also be of use in the methods of the invention. In some cases, the sample taken from a subject may be processed such that the biological markers are digested into smaller fragments. Alternatively, smaller fragments may be naturally present in the sample. Accordingly, reference herein to observing or detecting the level of the one or more biological markers should be taken to include reference to observing or detecting the level or one or more fragment of the one or more biological markers.

Typically the method will involve taking one or more sample from a subject, detecting or determining the level of one or more biological marker in one or more sample and comparing the level of the one or more biological marker against one or more standards. The difference in the level of the one or more biological marker in the one or more sample compared to the standard would allow diagnosis of a cardiac ischemic event.

Typically, the one or more sample would be taken from a patient on presentation to a hospital.

Preferably, the one or more sample would be taken from between 5 minutes to 8 hours of symptom onset. The one or more sample may then be analyzed to identify and preferably give a quantitative value for each of the biological markers detected. Preferably the values would then be compared against one or more standard to diagnose the probability of a clinical event having occurred. In the instance where the diagnostic test indicates a clinical event, this may then prompt the administration of a treatment.

The method of the invention preferably allows for diagnosis of a recent cardiac ischemic event.

In one embodiment, multiple samples may be taken from the subject and analyzed to detect one or more of the biological markers at different time points. For example, one or more sample may be taken and the level of one or more biological markers detected on presentation of a subject at a hospital. Then, after a period of time, one or more other sample may be taken and analyzed to detect the same or different one or more markers. This could be repeated a number of times, as desired, to monitor the subjects symptoms and condition. The results from each repeat of sampling and detection may be analyzed individually or together, to assist in the diagnosis of the subject or monitor their condition.

The samples may be taken, for example, from 1 minute to 5 minutes apart. In other embodiments, the samples are taken 10 minutes apart, 15 minutes apart or 20 minutes apart. In other embodiments the samples may be taken 30 minutes, 40 minutes, 60 minutes, 2 hours, 3 hours, 4 hours, 5 hours, 6 hours or 7 hours apart.

As used herein the terms “diagnosis”, “diagnostic” and the like should be taken broadly to include identifying the likelihood that a cardiac ischemic event has occurred as opposed to another event such as a pulmonary embolism, esophageal spasm, gastritis, gastric ulcer, pneumonia, gastroesophageal reflux or musculoskeletal pain. It should not be taken to be limited to a definitive diagnosis.

As used herein a “cardiac ischemic event” should be taken broadly to refer to any conditions in which coronary arteries have restricted blood flow, are obstructed, or are blocked or occluded, at least temporarily. “Cardiac ischemic events” include, for example, acute coronary syndrome, heart attack, myocardial infarction (including both myocardial infarction with the ST segment of an ECG elevated or not elevated), and angina.

As used herein a “recent cardiac ischemic event”, is one which has occurred within a period of approximately 5 minutes to approximately 8 hours from the time the one or more sample is taken from a subject; i.e. the event took place from approximately 5 minutes to approximately 8 hours before the sample was taken. In certain embodiments, the cardiac ischemic event has occurred within a period of approximately 10 minutes, 20 minutes, 30 minutes, 40 minutes, 60 minutes, 2 hours, 3 hours, 4 hours, 5 hours, 6 hours or 7 hours.

As used herein the term “long chain fatty acid” refers to any fatty acid with an aliphatic tail that is 12 carbon atoms or greater, including very long chain fatty acids. The long chain fatty acid can be either saturated or unsaturated Long chain fatty acids include, for example, oleic acid (C18:1), palmitic acid (C16:0) , palmitoleic acid (C16:1), stearic acid (C18:0), pentadecanoic acid (C15:0) and myristic acid (C14:0).

A “subject” as used herein is a mammal, preferably a human.

The diagnostic method of the invention can be practiced on any appropriate sample from a subject. However, by way of example, the sample can be a breath sample, a blood sample, including a plasma or serum sample, or a urine sample. In one particular embodiment, the sample is a breath sample.

In one embodiment, the method comprises detecting the level of one or more biological markers in each of two or more biological samples from the subject. In other embodiments, the method comprises detecting the level of one or more biological makers in each of three or more, or four or more biological samples taken from a subject. In one embodiment, the two or more samples are different. In another embodiment, the two or more samples are the same. In one embodiment, two or more samples may be taken at different time points.

By performing the methods of the invention on different samples from the same subject, a more rapid diagnosis is possible in a shorter time frame from the onset of symptoms.

In one embodiment, the samples may be taken from 1 minute to 5 minutes apart. In other embodiments, the samples are taken 10 minutes apart, 15 minutes apart or 20 minutes apart. In another embodiment the samples may be taken 30 minutes, 40 minutes, 60 minutes, 2 hours, 3 hours, 4 hours, 5 hours, 6 hours or 7 hours apart. Taking samples over an extended period may allow the subjects condition and/or treatment to be monitored.

Such samples can be taken from the patient using standard techniques known in the art. However, by way of example blood samples can be extracted from a vein using a needle, or by a finger prick. In one embodiment, breath samples may be analyzed using for example, selected ion flow tube mass spectrometry (SIFT-MS). However, breath samples may be taken by having the subject blow into a bag consisting of mylar or other inert substances for later analysis.

The diagnostic methods of the invention involve detecting the levels of one or more specific biological marker in a sample taken from a subject. The inventor(s) contemplate the specific biological marker mentioned herein to be useful as individual markers or in combination with each other. The inventors also believe that the levels of specific long chain fatty acids and CD44 relative to one another are useful in the diagnostic methods of the invention.

In one embodiment, myristic acid is detected. In another embodiment, oleic acid is detected. In another embodiment, both myristic and oleic acid are detected. In other embodiments, combinations of two or more of any of oleic acid (C18:1), palmitic acid (C16:0), palmitoleic acid (C16:1), stearic acid (C18:0), pentadecanoic acid (C15:0) and myristic acid (C14:0) are detected.

In another embodiment CD44 is detected alone or in combination with one or more of the long chain fatty acid biological markers.

In another embodiment, the method comprises detecting the level of both CD44 and oleic acid. In another embodiment, the method comprises detecting the level of both CD44 and myristic acid. In another embodiment, the method comprises detecting the level of CD44, oleic acid and myristic acid.

The sample may be processed prior to detecting the one or more biological marker to facilitate detection and/or analysis thereof. Skilled persons will appreciate appropriate processing steps and techniques suitable for performing them.

While it may be preferable to process a sample for diagnosis as quickly as possible so that diagnosis occurs early, it should be appreciated that samples may be taken and stored for a period of time before analysis. For example, blood samples can be collected using standard techniques and freeze dried. Samples can then be vortexed and centrifuged with the supernatant collected and stored at −80° C. prior to analysis. Breath samples can be stored in bags consisting of mylar or other inert substances for later analysis.

In one particular embodiment, samples may be processed prior to analysis in accordance with the methodology described in Villas-Boas, S. G., et al., Simultaneous analysis of amino and nonamino organic acids as methyl chloroformate derivatives using gas chromatography-mass spectrometry. Anal Biochem, 2003. 322(1): p. 134-8. See also Villas-Boas, S. G. and P. Bruheim, Cold glycerol-saline: the promising quenching solution for accurate intracellular metabolite analysis of microbial cells. Anal Biochem, 2007, 370(1): 87-97.

The diagnostic methods of the invention may involve comparing the level of the one or more biological marker against the level of the one or more biological marker in one or more standard. The difference in the level of the one or more biological marker in one or more sample compared to one or more standard being indicative of a cardiac ischemic event. The diagnostic methods of the invention may also involve comparing the level of the one or more biological marker against the level of other biological markers in the same sample taken from a subject, wherein the levels of the one or more biological marker relative to each other is indicative of a cardiac ischemic event. In one embodiment, the method involves comparing the level of one or more long chain fatty acid against the level of other long chain fatty acids in the sample, wherein the levels of the one or more long chain fatty acids relative to each other is indicative of a cardiac ischemic event.

In one embodiment, the one or more standard is a level of one or more of the biological markers known to be associated with substantially no history and/or evidence (symptoms or signs) of a cardiac ischemic event. In another embodiment, the one or more standard may be a level of one or more of the biological markers known to be associated with presentation of a cardiac ischemic event. The one or more standard may represent a mean value taken from a group or population of individuals or may be a value taken from an individual patient's medical history at one or more time when they have presented with a cardiac ischemic event or at one or more time when they have had no evidence of a cardiac ischemic event. In another embodiment the one or more standard is predicted by a model of the patients metabolism based on their genomic profile.

Preferably, the one or more standard comprises a control sample having a known level of one or more of the biological markers which is tested concurrently with the one or more samples from a subject in accordance with the invention. However, in another embodiment, the one or more standard could be a printed chart or electronic information or the like containing previously generated data considered to provide appropriate standards (as herein before described) and which test samples could be compared to. In one embodiment, such standards may be referred to as reference standards.

It should be appreciated that in addition to the samples and standards mentioned herein before, the method may include the testing of one or more positive or negative control samples to ensure the integrity of the results. For example, one could include a sample containing none of the biological markers and one or more samples containing a known level of one or more of the biological markers so that results can be calibrated across different runs of the method.

The one or more biological markers may be detected and the levels thereof compared to a standard using any one or a combination of techniques which are of use in identifying, quantifying and/or highlighting differential levels of fatty acids or proteins as the case may be. Such techniques will be readily appreciated by persons of ordinary skill in the art to which the invention relates. However, by way of example the one or more long chain fatty acids may be detected by mass spectrometry (GC-MS, LC-MS, MSMS), electrochemistry, and by the use of chemoresistive nanopolymers. In another example, the one or more long chain fatty acids are detected using selected ion flow tube mass spectrometry (SIFT-MS).

SIFT-MS is a real time mass spectrometry method pioneered in New Zealand (Syft Technologies), (see for example EP1540696 A1 and U.S. Pat. No. 7,429,730). This method is particularly good for detection of volatile fatty acids and is not currently used in cardiology. The nanopolymer sensor makes this a handheld technology that does not require the high voltage vacuum chambers that are needed in other MS equipment. SIFT is instantaneous, in real time, and direct from the subject.

CD44 may be detected by mass spectrometry or immunological techniques such as immunoassays including but not limited to enzyme linked immunosorbent assay (ELISA) (sandwich ELISA, double sandwich ELISA, direct ELISA, microparticle ELISA), radioimmunoassay (RIA), immunoprecipitation, Western blotting, immunohistochemical staining, or agglutination assay Protocols for carrying out such techniques are readily available; for example, see “Antibodies a Laboratory Manual”, Cold Spring Harbor Laboratory Press (1988), or the protocols described herein after. By way of further example, CD44 can be detected using antibody or aptamer based nanoassays such as those provided by rHealth (https://technology.grc.nasa.gov/SS-rHealth.shtm) and Nanosphere (www. nanosphere.us). In another example, CD44 can be detected indirectly using the ligand hyaluronic acid. In another example, CD44 can be detected indirectly by detecting mRNA of the CD44 protein.

The nanoassays of Nanosphere, rHealth as mentioned above are more sensitive and faster than traditional methods.

In one embodiment of the invention, breath samples are used for detection of small molecules and fatty acids, and blood samples are used for detection of larger proteins such as troponin and CD44.

In one embodiment of the invention, breath samples can be analyzed using selected ion flow tube mass spectrometry (SIFT-MS).

In one embodiment of the invention, blood samples can be analyzed either at the point of care or at a remote location using a hand held device as described, for example, in U.S. patent application Ser. No. 13/374,683.

In one embodiment, the methods of the invention may combine the use of two or more detecting techniques. In one embodiment, two or more techniques are used to analyze the same biological marker and/or the same sample. Where more than one biological marker is to be detected or more than one sample is to be analyzed, one or a combination of detection techniques may be used. The samples may be analyzed simultaneously or sequentially in any order. Combining detecting techniques may increase the accuracy of results.

In one embodiment, breath and blood samples are analyzed simultaneously.

The samples can be analyzed simultaneously using devices such as those provided by Scanadu (www.scanadu.com/), Tricorder (www.tricorderproject.org/)rHealth (https://technology.grc.nasa.gov/SS-rHealth.shtm) and Nanosphere (www.nanosphere.us).

The difference in the levels of the one or more of the biological markers in a sample versus a standard may be compared using standard technology having regard to the methods employed to detect the one or more long chain fatty acids and/or CD44 or any one or more other markers that may be detected in a method of the invention.

Classification methods for discriminating differences in markers compared to a standard and to determine whether an event has occurred include but are not limited to principal component analysis, partial least squared regression, soft independent modelling of class analysis and support vector machine learning.

When referring to the level of one or more biological marker from a subject compared to one or more standard, the terms “higher”, “lower”, “increased” and “decreased” and like terms may be used. Such terms should be taken broadly to include any change in the level of a biological marker in a sample, compared to a standard. However, in one embodiment, there is at least an approximately 1.5 fold difference in the level of the one or more biological markers, more preferably at least an approximately 2 fold difference, compared to the standard. In another embodiment, there is at least an approximately 5 fold difference in the level of the one or more biological markers compared to the standard.

In one embodiment, a standard is a level of the one or more biological markers which is associated with the absence of a cardiac ischemic event and a higher level of any one or more biological markers in any one or more sample is indicative of presenting with a cardiac ischemic event and a lower level or substantially the same level in one or more sample is indicative of the absence of a cardiac ischemic event. In another embodiment a higher level of any 2 or more, 3 or more, 4 or more, 5 or more, or 6 or more markers is indicative a cardiac ischemic event and any lower level or substantially the same level of any 2 or more, 3 or more, 4 or more, 5 or more, or 6 or more markers is indicative of the absence of a cardiac ischemic event. In another embodiment, where two or more samples are analyzed, a higher level of any 1 or more, any 2 or more, 3 or more, 4 or more, 5 or more, or 6 or more markers in at least two or more samples is indicative of presenting with a cardiac ischemic event and a lower level or substantially the same level of any 1 or more, any 2 or more, 3 or more, 4 or more, 5 or more, or 6 or more markers in two or more samples, is indicative of the absence of a cardiac ischemic event. In another embodiment, where three or more samples are analyzed, a higher level of any 1 or more, any 2 or more, 3 or more, 4 or more, 5 or more, or 6 or more markers in at least three or more samples is indicative of presenting with a cardiac ischemic event and a lower level or substantially the same level of any 1 or more, any 2 or more, 3 or more, 4 or more, 5 or more, or 6 or more markers in three or more samples, is indicative of the absence of a cardiac ischemic event. In another embodiment, where four or more samples are analyzed, a higher level of any 1 or more, any 2 or more, 3 or more, 4 or more, 5 or more, or 6 or more markers in at least four or more samples is indicative of presenting with a cardiac ischemic event and a lower level or substantially the same level of any 1 or more, any 2 or more, 3 or more, 4 or more, 5 or more, or 6 or more markers in four or more samples, is indicative of the absence of a cardiac ischemic event.

In another embodiment, a standard is a level of the one or more biological markers which is associated with a cardiac ischemic event and a level of the one or more biological markers in any one or more sample which is substantially the same as or higher than a standard is indicative of a cardiac ischemic event and a lower level of any one or more biological marker in any one or more sample compared to a standard is indicative of the absence of a cardiac ischemic event. In another embodiment, a level of two or more, three or more, four or more, five or more, or six or more markers which is substantially the same as or higher than a standard is indicative of a cardiac ischemic event and a lower level of any two or more, three or more, four or more, five or more or six or more markers is indicative of the absence of a cardiac ischemic event. In another embodiment, where two or more samples are analyzed, a level of any one or more, 2 or more, 3 or more, 4 or more, 5 or more, or 6 or more markers in at least two or more samples which is substantially the same as or higher than a standard is indicative of a cardiac ischemic event and a lower level of any one or more, 2 or more, 3 or more, 4 or more, 5 or more, or 6 or more markers in at least two or more samples compared to a standard is indicative of the absence of a cardiac ischemic event. In another embodiment, where three or more samples are analyzed, a level any one or more, 2 or more, 3 or more, 4 or more, 5 or more, or 6 or more markers in at least three or more samples which is substantially the same as or higher than a standard is indicative of a cardiac ischemic event and a lower level of any one or more, 2 or more, 3 or more, 4 or more, 5 or more, or 6 or more markers in at least three or more samples compared to a standard is indicative of the absence of a cardiac ischemic event. In another embodiment, where four or more samples are analyzed, a level of any one or more, 2 or more, 3 or more, 4 or more, 5 or more, or 6 or more markers in at least four or more samples which is substantially the same as or higher than a standard is indicative of a cardiac ischemic event and a lower level of any one or more, 2 or more, 3 or more, 4 or more, 5 or more, or 6 or more markers in at least four or more samples compared to a standard is indicative of the absence of a cardiac ischemic event.

In particular preferred embodiments there is at least an approximately 1.5 fold increase or decrease of oleic acid, myristic acid, palmitic acid, palmitoleic acid, stearic acid, and/or pentadecanoic acid compared to a standard is diagnostic of a cardiac ischemic event. In another particular preferred embodiment there is at least an approximately 2 fold increase or decrease of oleic acid, myristic acid, palmitic acid, palmitoleic acid, stearic acid, and/or pentadecanoic acid compared to a standard is diagnostic of a cardiac ischemic event.

In another embodiment, the one or more biological markers is CD44 and there is at least an approximately 5 fold difference in the level of the one or more biological markers compared to a standard.

In one embodiment, the diagnostic methods of the invention can be integrated and implemented with computer systems, software and processes, as can the results of the diagnostic methods of the invention.

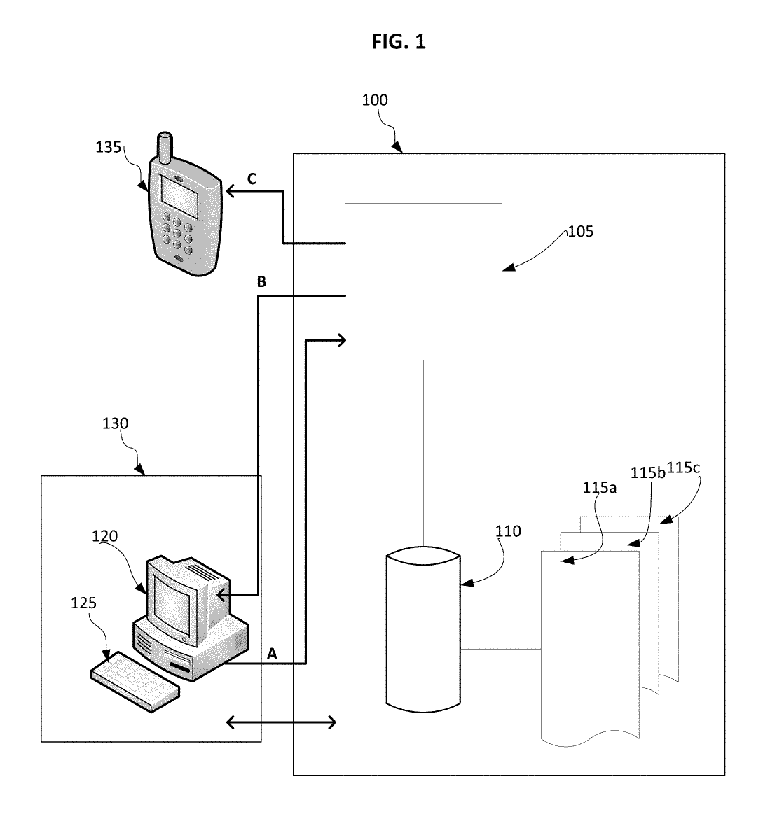

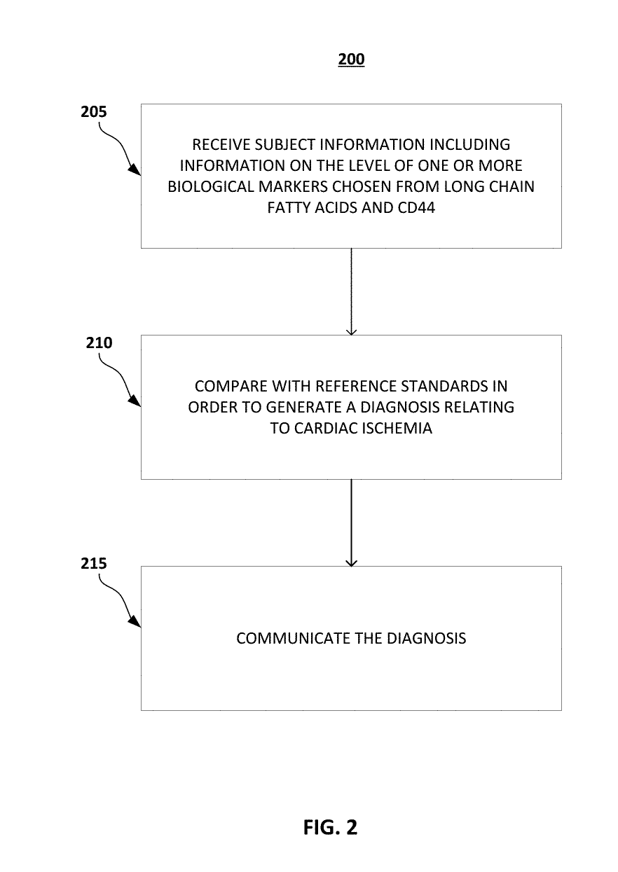

One embodiment is shown with respect to FIGS. 1 and 2. FIG. 1 is a schematic diagram of a system for diagnosis in relation to cardiac ischemia in a subject. The system 100 comprises a processor 105 coupled to a memory 110 having one or more reference standards 115A. These reference standards may include a level of one or more biological marker comprising one or more long chain fatty acid and CD44 associated with cardiac ischemia, or the level of the one or more biological markers which indicate the absence of a cardiac ischemic event. The memory may include one or more of standard ECG data and/or ultrasound data 115C, standard information on the level of one or more additional biomarkers associated with cardiac ischemic or the absence of a cardiac ischemic event, historical clinical observations, medical history, genomic profile, family history, previous test results and other medically relevant data. The processor 105 and memory 110 may be any suitable components such as those found in a personal computer or the like. The memory 110 may also contain a computer program 115B which when executed on the processor 105 causes the processor to behave as a diagnostic system as described in more detail below.

A user interface 130 is coupled to the diagnostic system 100, and comprises a display 120 and a keyboard and/or mouse combination for entering data into the diagnostic system 100. Alternative input systems may be used, for example handheld patient testing devices and the like which may communicate with the system 100 via electrical connection or wirelessly. There may also be provided a remote device 135 such as a mobile phone for receiving information from the diagnostic system 100.

FIG. 2 is a flow diagram illustrating a computerized method for diagnosing in relation to cardiac ischemia in a subject, and which may be implemented in the system of FIG. 1. In the method 200, the diagnostic system 100 receives a subject information (including information on the level of one or more biological markers chosen from long chain fatty acids and CD44) for a subject at step 205. This subject information may additionally include other data such as ECG data, ultrasound data, the level of one or more additional biomarkers, clinical observations and/or medical history. This subject information may be received from the user interface 130 in response to user interaction with the keyboard 125 alternatively this information may be received from a data storage media such as a USB stick or via a communications link from a remote device.

The method then compares the received subject information with one or more reference standard in order to generate a diagnosis relating to cardiac ischemia, as indicated at step 210. The diagnosis may be generated by any suitable algorithm using a comparison of the level of the one or more biological markers (including one or more long chain fatty acid levels and/or CD44 levels) received in the subject information, and the same one or more biological marker from the one or more reference standard. In an embodiment, the diagnosis is that the subject has had a cardiac ischemic event if the level of the one or more biological markers in the received subject information is higher than the levels in the reference standard. The diagnosis may alternatively be that there has been no cardiac ischemic event.

In step 215 the method communicates the diagnosis. This may be implemented by the processor 105 causing the display 120 of the user interface 130 to display the diagnosis. In another implementation the processor may cause the transmission of the diagnosis to a remote device 135 such as a mobile phone. In a further implementation the processor 105 may activate a visual and/or audial alarm indicating that the subject has had a cardiac; ischemic event for example.

The method and system may be further arranged to generate and communicate an updated diagnosis in response to receiving updated subject information for the subject. This subject information may be regularly updated by a nurse or an automated diagnostic tool in communication with the diagnostic system 100. The updated diagnoses may indicate one or more cardiac ischemic events or they may be used to indicate subject response to treatment by monitoring rising or falling levels of the one or more biological markers of the invention over time alone or in combination with other subject information. For example a warning may be communicated when these levels rise over a threshold and/or rise sufficiently quickly. Similarly an indication may be communicated that the subject is responding to treatment when the level of one or more of the biological markers of the invention continue falling over a period of time alone or in combination with other subject information.

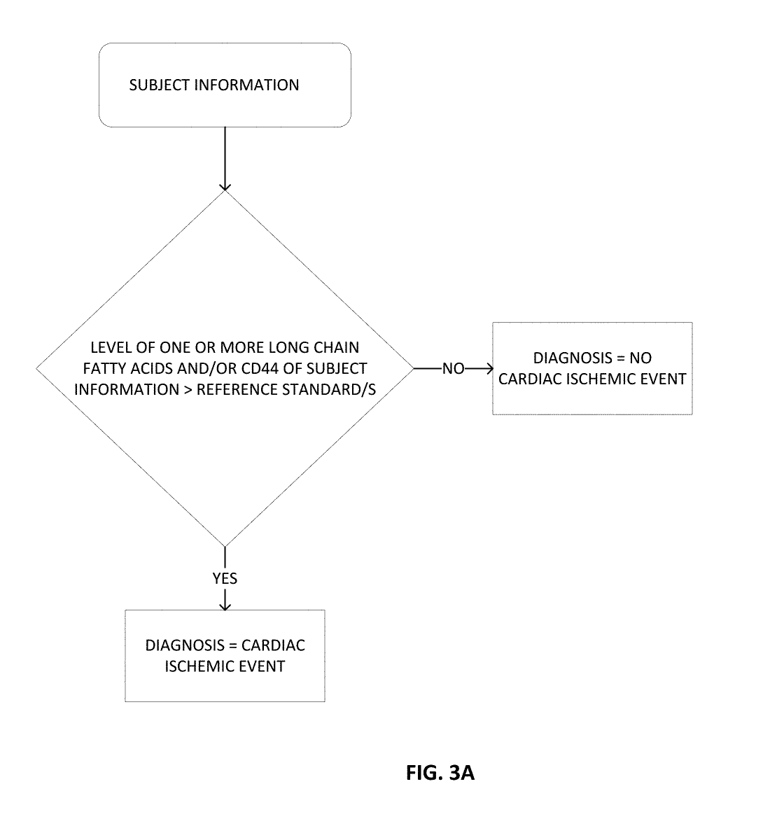

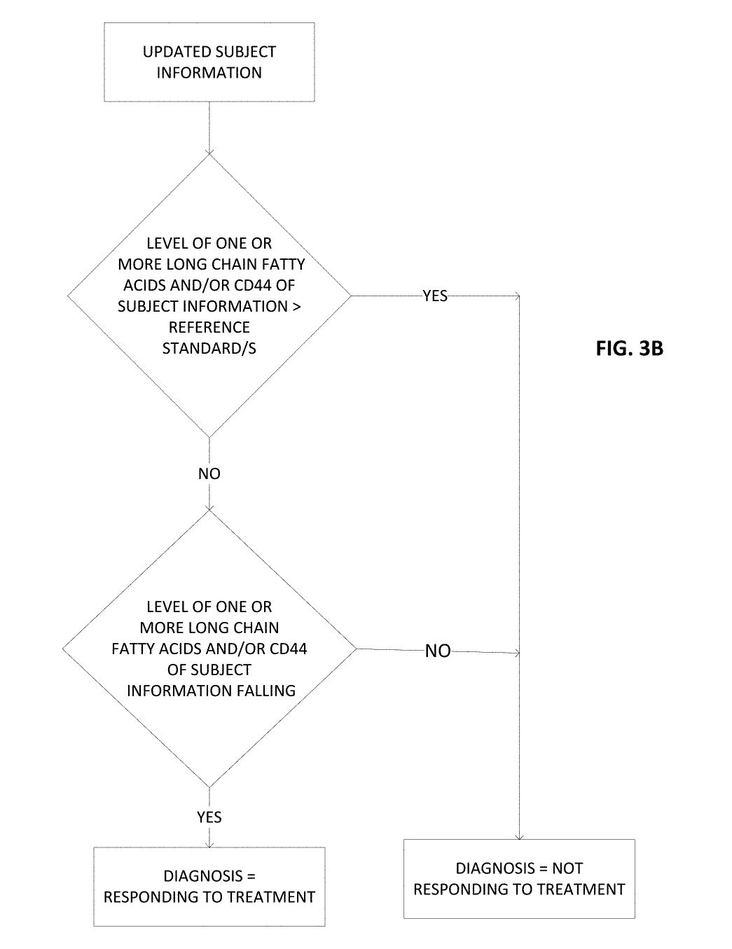

FIGS. 3a and 3b are flow diagrams illustrating algorithms for generating a diagnosis. Although specific algorithms for generating a diagnosis have been described and illustrated, this should not be construed as limiting. Various other algorithms consistent with the diagnostic teachings herein may be implemented using the described system 100 and method 200 in order to generate appropriate diagnoses for communication as would be understood by those skilled in the art.

Although the computer program 115B has been described as being contained in the same memory as the standard reference 115A, this computer program may also exist outside the system 100, for example on non-transitory storage media such as a CD ROM or a USB memory stick for example. The computer program may also be downloaded for example over the internet or using an electromagnetic signal.

It should be appreciated that the methods of the invention may also include analysis of or detecting the level of one or more other biological markers or clinical observations which are known to be associated with cardiac ischemic events. For example, detection or analysis of the levels of troponin I, trophonin T, creatine kinase myoglobin (CK-MB), myeloperoxidase and lactate dehydrogenase (LDH, LDH1 and LDH2). Additional biological markers include aspartate aminotrasferase, lactate dehydrogenase, creatine kinase, hydroxybutyrate dehydrogenase CK-MB (activity), CK-MB (mass), CK isoforms, myoglobin, carbonic anhydrase Ill, glycogen phosphorylase BB, Heart fatty acid binding protein, myosin light chains, pregnancy-associated plasma protein, choline, ischemia-modified albumin, unbound free fatty acids, placental growth factor, myeloperoxidase, MMP-9, sCD40L and troponin I or T. Skilled person will readily appreciate what the presence, absence or an increase or decrease in these markers is indicative of.

However, previously not all of these markers have been used in a clinical setting. Markers typically used in a clinical setting include CK-MB, CK, troponin I, troponin T, LDH, aspartate aminotransferase and myeloperoxidase. In one embodiment, performing an ECG and/or ultrasound and analyzing results in combination with detecting the level of one or more biological marker of the invention is also envisaged.

The inventors have also identified the following biological markers that can be detected in addition to the biological markers of the invention: tryptophan, glycine, lysine, isoleucine, leucine, hydroxybutyric acid, phenylalanine, valine, creatinine, threonine, aspartic acid, glutamic acid, pyroglutamic acid, alanine, cysteine, and lactic acid. The inventors have observed that the presence of a change in the level of the one or more of these markers compared to one or more standards is indicative of a cardiac ischemic event.

The inventors also envisage that different markers can be detected in combination. The inventors believe that the best combinations are any combinations of the biological markers myristic acid and/or oleic acid and/or CD44 in combination with one or more of the following: tryptophan, glycine, lysine, isoleucine, leucine, hydroxybutyric acid, phenylalanine, valine, creatinine, threonine, aspartic acid, glutamic acid, pyroglutamic acid, alanine, cysteine, and lactic acid and troponin I or T. In one particular embodiment the methods of the invention involve detecting myristic acid and/or oleic acid and/or CD44 in combination with Troponin I and/or Troponin T.

In a particular embodiment, troponin I measurement is possible on the Nanosphere analyzer mentioned herein.

The inventors consider that analysis of one or more additional biological markers and/or making further clinical observations (including ECG and/or ultrasound results) in addition to detecting the biological markers of the invention will increase the sensitivity and specificity of the methods of the invention when making a diagnosis. In particular, combining the analysis of one or more of the biological marker of the invention and electrocardiography and/or ultrasound may provide a more accurate diagnosis.

The inventors note that analysis of biological markers, ultrasound and/or electrocardiography measurements can be taken rapidly at the bedside. However, such techniques have not previously been integrated as detailed herein. This will provide significant benefits in rapid and accurate point of care diagnosis.

Detection of one or more other biological marker and/or ECG and/or ultrasound can be performed using standard method known in the art. However, by way of example, one or more biological marker may be detected using nuclear magnetic resononance (NMR) spectrometry and gas chromatography mass spectrometry (GC-MS). For ECG and ultrasound imaging spectral components of these modalities would include ultrasound derived global myocardial longitudinal strain and advanced electrocardiography derived scores (U.S. Pat. Nos. 7,539,535 and 7,386,340), for example.

In one embodiment, the method of the invention further comprises the step of treating a subject for cardiac ischemia where a method of the invention indicates diagnosis of a cardiac ischemic event.

In another embodiment, the method of the invention further comprises the step of deciding not to treat a subject for cardiac ischemia where a difference in the level of the one or more biological markers in the sample compared to the standard is not diagnostic of a cardiac ischemic event.

In a related embodiment, the methods of the invention can also be used for monitoring a patients response during or after treatment, as herein before described.

As mentioned herein before, preliminary studies by the inventor(s) indicate that myristic acid, oleic acid, and other long chain fatty acids may be used for the treatment of cardiac ischemia and PPAR-related disorders.

As used herein, the term “PPAR” refers to any one of the following peroxisome proliferator-activated receptors: PPAR-a, PPAR-o, or PPAR-y.

As used herein, the terms “PPAR related illness” or “PPAR related disease” or like terms are intended to refer to any illness, disease, or disorder where the condition can be treated by activation of PPAR.

Disorders include, for example those associated with aberrant cellular differentiation or growth, metabolism, inflammation or tumorogenesis.

Accordingly, in another aspect of the invention, there is provided a pharmaceutical composition comprising one or more long chain fatty acids or one or more salt thereof optionally in combination with one or more pharmaceutically acceptable diluents, carriers and/or excipients.

Preferably, the long chain fatty acids are selected from myristic acid, oleic acid, palmitic acid, palmitoleic acid, stearic acid, and pentadecanoic acid. In a preferred embodiment pharmaceutical composition comprises myristic acid, oleic acid or a combination of both.

In one embodiment, such disorders include cardiac ischemic events, atherosclerosis, diabetes mellitus, malaria and various forms of cancer. In a particularly preferred embodiment, the disease is a cardiac ischemic event.

As used herein, the phrase “pharmaceutically acceptable diluents, carriers and/or excipients” is intended to include substances that are useful in preparing a pharmaceutical composition, may be co-administered with an active agent of the invention while allowing it to perform its intended function, and are generally safe, non-toxic and neither biologically nor otherwise undesirable. Pharmaceutically acceptable diluents, carriers and/or excipients include those suitable for veterinary use as well as human pharmaceutical use. Examples of pharmaceutically acceptable diluents, carriers and/or excipients include solutions, solvents, dispersion media, delay agents, emulsions and the like.

Those of ordinary skill in the art will readily appreciate a variety of pharmaceutically acceptable diluents, carriers and/or excipients which may be employed in compositions of the invention. However, by way of example, suitable liquid carriers, especially for injectable solutions, include water, aqueous saline solution, surface polymers such as pluronic F-127, aqueous dextrose solution, and the like, with isotonic solutions being preferred for intravenous administration.

In addition to standard diluents, carriers and/or excipients, a pharmaceutical composition comprising an active agent in accordance with the invention may be formulated with additional constituents, or in such a manner, so as to enhance the activity of the active agent, target the composition to a particular cell type, facilitate cell permeability, help protect its integrity, or extend its half life, for example. This may include coatings, or formulating the composition in a micelle, as is described herein after. The composition may further comprise additional active ingredients other than the one or more long chain fatty acids which may have a further therapeutic benefit.

The pharmaceutical compositions of the invention may be formulated into any appropriate dosage form using standard methodology known in the art. However, by way of example, the compositions may be in the form of injectable liquids, orally administrable liquids, tablets, coated tablets, capsules, pills, granules, suppositories, trans-dermal patches, suspensions, emulsions, sustained release formulations, gels, aerosols, and powders may be used. Skilled persons will readily recognize appropriate formulation methods. However, by way of example, certain methods of formulating compositions may be found in references such as Gennaro AR: Remington: The Science and Practice of Pharmacy, 20th ed., Lippincott, Williams & Wilkins, 2000.

Another aspect of the invention is a method of treating a disorder as mentioned herein before comprising the step of administering a pharmaceutical composition of the invention to a subject.

As used herein, the term “treatment” is to be considered in its broadest context. The term does not necessarily imply that a subject is treated until total recovery. Accordingly, “treatment” includes amelioration of the symptoms or severity of a particular condition or preventing or otherwise reducing the risk of developing a particular condition.

The composition is preferably administered to a subject after diagnosis of a relevant illness.

The composition may be administered to a subject via any conventional route of administration having regard to the nature of the disease to be treated and the dosage form of the composition. However, by way of example, administration methods may include parenteral administration, systemic administration, oral and topical administration. The term “parenteral” is intended to refer to subcutaneous-, intracutaneous-, intravenous-, intramuscular-, intraarticular-, and intraaterial-injection, for example.

As will be appreciated, the dose of an agent or composition administered, the period of administration, and the general administration regime may differ between subjects depending on such variables as the severity of symptoms of a subject, the condition to be treated, the mode of administration chosen, and the age, sex and/or general health of a subject. However, by way of general example, the inventor(s) contemplate administration of from approximately in g/kg to 1 g/kg of active agent per body weight of the subject to be treated. More preferably, the range is 1 ng/kg to 500 mg/kg, even more preferably, 10 mg/kg to 300 mg/kg of the body weight of the subject to be treated.

It should be appreciated that administration may include a single daily dose or administration of a number of discrete or divided doses as may be appropriate.

It should be appreciated that a method of the invention as above mentioned may further comprise additional steps such as the administration of additional agents or compositions which may be beneficial to a subject, concurrently or sequentially, in any order. Similarly, the method may involve administering more than one composition of the invention, wherein the compositions contain different active ingredients (for example, oleic and myristic acid, palmitic acid, palmitoleic acid, stearic acid, pentadecanoic acid), concurrently or sequentially, in any order.

In one embodiment, a pharmaceutical composition of the invention may be formulated for targeted delivery to a particular cell type or region of the subject's body. In one embodiment, the composition may be formulated in the form of a micelle which is preferably adapted for targeted delivery to a cell.

In one embodiment, the micelle comprises an amphiphilic co-polymer in combination with one or more cell targeting molecules.

The amphiphilic co-polymer may be any suitable co-polymer as will be appreciated by persons of ordinary skill in the art.

The cell targeting molecule is any molecule which allows the micelle to target delivery to a particular cell type. By way of example, in one embodiment the cell targeting molecule is a ligand capable of binding to a molecule, such as a receptor, on the surface of a target cell to which the composition of the invention is to be delivered. It should be appreciated that any suitable receptor-ligand interaction known in the art can be used, having regard to the nature of the cells to be targeted.

Preferably, the receptors are selected from integrins (alpha-v, beta-3 integrin), selectins, vascular cell adhesion molecule (VCAM)-1, intercellular adhesion molecule 1 (ICAM-1), PECAM 1, junction adhesion molecules (JAMs), connexins, CD44 (cluster of differentiation 44), and CD36 (cluster of differentiation 36).

In one preferred embodiment, the ligand is hyaluronic acid and the receptor is CD44.

In other embodiments, the receptor is CD44 and the ligand is chosen from collagen, laminin, fibronectin and osteopontin.

In another preferred embodiment the receptor is CD36 and the ligand may be chosen from collagen, thrombospondin, erythrocytes parasitized with Plasmodium falciparum, oxidized low density lipoprotein, native lipoproteins, oxidized phospholipids, and long-chain fatty acids.

The micelle can also comprise detection agents for visualizing and detecting delivery of the active ingredients of the invention. For example, colorimetric and fluorometric techniques may be used in which a detection molecule is labelled with a molecule which can be visualized by the naked eye or otherwise detected using a spectrophotometer, or fluorometer for example. Alternatively, detection molecules could be labelled with radio-isotopes. Alternatively, detection can be visualized using quantum dot technology. The quantum dots can comprise silicon.

Methods for labelling and subsequently measuring the intensity of signals generated will be known to those of skill in the art to which the invention relates. However, by way of example techniques that can be used include Fourier transform near infrared (FTIR) spectroscopy, Raman spectroscopy, and near infrared spectroscopy. In one embodiment the quantum dots comprising silicon are imaged with a cellular phone camera. In another embodiment the quantum dots are imaged using MM, or SQUID (Superconducting Quantum Interfering Device) based low field MRI.

Micelles and labelling means may be prepared using methods known in the art. However, by way of example see, Erogbogbo et al, ACS NANO, Vol. 4, No. 9, (2010), 5131-5138 and Erogbogbo et al, ACS NANO, Vol. 2, No. 5, (2008), 873-878.

The invention also provides micelles comprising a cell targeting molecule and containing one or more pharmaceutical compounds or compositions.

The micelle preferably comprises an amphiphilic co-polymer.

The cell targeting molecule may be as herein before described.

The pharmaceutical composition may be a pharmaceutical composition of the invention or any other appropriate composition. In one embodiment, the pharmaceutical compound or composition comprises dexamethasone.

The micelle may also comprise one or more other compounds. By way of example only, it may include one or more agents for visualizing and detecting delivery as herein before described.

The inventors have identified that it is possible to target cardiac cells for delivery of a compound or agent by targeting CD44 Accordingly, the invention also provides the use of a CD44 ligand to target an agent for delivery to cardiac cells, circulating peripheral blood cells, endothelial cells, multipotent haemopoietic stem cells or rare circulating cells. The ligand may be combined with the agent in any suitable form including, being connected or fused to the agent, or provided on the surface of a formulation comprising the agent, as is described for the micelle above. Accordingly, the invention also provides constructs and compositions comprising an agent to be delivered to a cardiac cell, circulating peripheral blood cell, endothelial cell, multipotent haemopoietic stem cell or rare circulating cell and a ligand for CD44.

The invention also relates to a kit of use in a method of the invention, the kit comprising at least one or more reagents suitable for detection of the one or more biological markers as herein before described.

Reagents of use in processing samples for analysis may also be contained in the kits of the invention. They may also comprise one or more standard and/or other controls containing known levels of the one or more biological markers in accordance with the invention. Further, kits of the invention can also comprise instructions for the use the components of the kit as well as printed charts or the like that could be used as standards against which results obtained from test samples could be compared. Reagents may be held in any suitable container.

### EXAMPLE

A study was performed to determine the metabolomic profile of plasma taken from the coronary sinus of patients immediately prior to coronary angioplasty and at several subsequent time points following the procedure. The purpose for this was to characterize mechanistic alterations in cardiac metabolism at the time of percutaneous coronary intervention (PCI). The hypothesis was that this method would identify alterations in pathways related to cardiac energetics, apoptosis due to ischemia, coagulation, inflammation and preconditioning. A targeted approach using GC-MS was undertaken in an attempt to determine metabolomic markers.

33 patients gave informed consent to be enrolled into the trial. Patients with a first non ST elevation myocardial infarction and serum troponin T>0.1 mmol/l undergoing coronary angiography between 24 hours and six days following were screened. Eligible patients with a single identifiable culprit lesion either of the left anterior descending (LAD) or dominant right coronary artery (RCA) and who were to undergo adhoc PCI were randomized.

A double blinded 2:1 LAD to RCA randomization through a pseudorandom number generation program was followed. Patients were also randomized 1:1 to receive intracoronary metoprolol at the beginning of the procedure.

Exclusion criteria included acute myocardial infarction within the precedin 24 hours, haemodynamic instability (including cardiogenic shock, systolic blood pressure <100 mmHg, uncontrolled heart failure or significant LV impairment (EF<35 percent))significant (moderate-severe) valvular disease, renal impairment (creatinine >0.16 rnmol/l), occluded vessel or extensive angiographic thrombus on diagnostic angiography and contraindication to beta blockade (including: asthma, current use of bronchodilator therapy, 2^{nd}/3^{rd} degree AV block, known sick sinus syndrome or baseline bradycardia <50 bpm).

At the time of the intervention the majority of patients were taking an oral beta-blocker, which was continued during the study. All non-study medications including use of heparins, direct thrombin inhibitors, clopidogrel and glycoprotein llb/Illa inhibitors were administered at the discretion of the leading physician.

### Study Procedure:

Baseline angiography was performed in the usual manner. A catheter was advanced through the coronary sinus and into the great cardiac vein to ensure selective sampling of LAD territory drainage. Catheter position was confirmed by contrast injection. Baseline blood samples were simultaneously taken from the great cardiac vein and ascending aorta. Angioplasty was then performed with a mandated initial predilatation of 60 seconds, unless otherwise indicated on clinical grounds. Beginning 10 seconds after the first balloon deflation a further blood sample from the great cardiac vein was taken. Following PCI and at least 20 minutes after the first and five minutes after last balloon inflation final blood sample from the great cardiac vein (CS), ascending aorta (AO) and femoral vein were drawn.

### Metabolomics Sample Preparation, Extraction and Analysis

Plasma was prepared for metabolomic analysis using the methods outlined by Smart et al (Nat Protoc. 2010 September; 5(10):1709-29).

Proteomics Substudy (n=B) Sample Preparation and Analysis:

Blood sampling was done at baseline (pre-PCI) and 18±2 min from the first balloon inflation (post-PCI), using multipurpose catheters placed in the coronary sinus. Blood was collected into 5 ml EDTA (ethylenediaminetetraacetic acid) vacutainer tubes (Becton, Dickinson and Company) and centrifuged at RT at 3310×g for five mins, within two minutes of collection. One ml of plasma was pipetted into microtubes containing pepstatin A (Sigma Aldrich) and bestatin (Sigma Aldrich) to give a final concentration of 8 pmole/L of pepstatin A and 16 pmole/L of bestatin. All samples were snap frozen in dry ice methanol slurry.

### Depletion of Plasma Samples:

To allow for detection of moderately abundant plasma proteins, plasma samples were depleted of the 12 most abundant proteins using the ProteomeLab IgY-12 High Capacity SC Spin Column Kit (Beckman Coulter), according to the instructions provided by the manufacturer. Depleted samples were stored at −80° C. until analyzed.

### Sample Preparation and Liquid Chromatography-Tandem Mass Spectrometry (L C-MSIMS):

Depleted samples were prepared for iTRAO labeling according to Jullig et al (Proteomics Clin Appl. 2007; 1:565-76 with some modification. Defrosted 2 ml samples were concentrated to 1 ml using a Savant SPD121P SpeedVac Concentrator (Thermo Savant, Holbrook, N.Y.), supplemented with dithiothreitol (DTT) to a final concentration of 10 mM and incubated at 57° C. for one hr. Iodoacetamide (IAM) was then added to 20 mM and the samples were incubated for at RT for one hr in the dark before inactivation of IAM by addition of excess DTT. Protein concentrations were determined using the Bradford method (BioRad). Sample volumes corresponding to lOOpg were taken to fresh low protein-binding microcentrifuge tubes (Axygen Inc., CA) and supplemented with 2 pg trypsin.

Digestion was performed at 37° C. for 16 hrs. Digested plasma protein from each preparation was then labelled with 4-plex iTRAQ reagent, according to the manufacturer's description. The labels were rotated between the runs to reduce potential labelling bias or interference. Prior to LC-MS/MS, four paired samples with different labels (pre-PC and corresponding post-PC from two patients were combined, allowing for analysis of all 16 patient samples over four separate LC-MS/MS runs. The combined pools of iTRAQ-Iabelled samples were then fractioned by on-line cation exchange using 15 salt steps and the resulting LC effluent was directed into the ion spray source of a QSTAR XL hybrid mass spectrometer (Applied Biosystems, Foster City, Calif.) set to scan from 300 to 1600 m/z and with the top three most abundant multiply charged peptides selected for MS/Manalysis (80-1600 m/z) Protein Pilot 1.0 software (Applied Biosystems, Foster City, Calif.) was then used with the “rapid” search effort to search the output data against the human I PI v3.27 database with carbamidomethyl cysteine as fixed modification and trypsin as the enzyme specificity. The ratio of false-positive identifications was estimated by performing an identical search of the data against the same database with all protein sequences reversed. In cases where the protein name in the database were insufficient (e.g. “unnamed protein”) a BLAST search (using the software available online) based on the amino acid sequence of the identified protein was made. In most cases it found an identical protein with an established name.

### Processing of the ProteinPilot Output:

Changes in relative protein abundance were assessed manually to avoid known software problems ProteinPilot peptide summaries for all matched peptides were initially saved and used to normalize the total level of iTRAQ labelling for each label within each run, after removal of any spectra where the peptides ended with a C-terminal proline, which is known to interfere with the 116 label (Jullig et al), all spectra shared between proteins, spectra matched with confidence=0, spectra matched to proteins with an unused score less than two, and all spectra with insufficient labelling (raw iTRAQ label area sum less than 40 for each two-sample comparison). The levels of residual depletion targets (expressed as the percentage of the total iTRAQ signal matched to depletion targets), was then assessed as a rough guide for depletion efficiency. For the 16 depleted samples, on average 31.1%±1.5% (S.E.M.) of the total iTRAQ signal originated from proteins that should theoretically no longer have been present in the samples. The most completely depleted sample contained 19.3 percent depletion targets while the least depleted sample contained 39.2 percent. In order to bypass variations introduced by uneven depletion efficiency, all spectra matched to any of the depletion targets were excluded prior to normalization, as were spectra matched to introduced pig trypsin. Given the observed tendency toward more missed cleavages in the post-PCI samples compared to pre-PCI samples normalization was performed using separate correction factor for spectra matched to correctly cleaved peptides and spectra matched to peptides with missed cleavages. Individual protein ratios (post-PCI versus pre-PCI) within each run were then calculated from log-transformed sums of the corrected raw area values for each protein. Finally, overall relative abundance (post-PCI versus prePCI) for each protein was calculated as the average of the previously obtained protein ratios (a maximum of eight ratios for each protein across four runs).

### Proteomic Data Analysis:

In order to evaluate changes at the individual level, an initial analysis was performed using log ratios of each patient's post-PCI versus pre-PCI values (average for each protein)obtained for all 77 proteins that were found in at least four comparisons, as described above. Significance was determined using a two-tailed Student's t-test assuming unequal variance, p<0.05 was considered significant and p<0.1 was considered trending. Further to this first pass analysis proteins found to change with p<0.1 0 were subjected to a second test in order to predict the relevance of these findings across patients, i.e. their potential usefulness as biological markers. For this, protein log ratios were obtained for each post-PCI versus the non-corresponding pre-PCI value analyzed within the same LC-MS/MS run. Significance was then determined using an unpaired two-tailed Student's t-test assuming unequal variance, p<0.05 was considered significant and p<0.1 was considered trending. In addition, due to the small sample size, the consistency of behavior in response to treatment was observed, along with the above mentioned statistical significance.

### Metabolomics Sample Analysis:

Raw files produced by Chemstation from the GC-MS analysis of the samples (Data Files .D files). The software PAPi was used to measure fragment ratios in peaks over their elution, which are consistent) and peak height to qualify and quantify the values using the methodology of Aggio, R., Ruggiero, K. and Villas-Boas, S. (2010) Pathway Activity Profiling (PAPi): from metabolite profile to the metabolic pathway activity. Bioinformatics. doi:10.1093/bioinformatics/btq567.

### Metaboloimic Data Analysis:

The metabolomic data was initially transformed using Principal Component Analysis (PCA) before bioinformatic methods were applied. A signal to noise (SNR) method was used to rank data from most important to least important based on SNR.

$$SNR ( X ) \begin{matrix}\vert Mean ( {x}_{a} - mean ( {x}_{b} ) \vert \\ std ( {x}_{a} ) + std ( {x}_{b} )\end{matrix}$$

(Where Xₐ are the values of variable X belonging to class A and X_{b} are the variables of X belonging to class B)

The data was pre-processed (missing values filled in, normalization) and then split several times (three to six folds cross validation) into a training part (e.g. 70 percent) and test part (e.g. 30 percent). Featured were extracted and ranked from the training part and then these features and the training data were used to build a personalized model (PM) for every sample from the test data. The accuracy of classification was established as average for all folds and a set of ranked features (potential global markers), that are mostly selected during the folds, was obtained. Methods included both inductive and transductive techniques. Six discriminatory techniques were used to separate the data. These techniques can be grouped into globalized, localized and personalized methods. The two globalized approaches used were; Multi-Linear Regression and Support Vector Machines. The two localized techniques used were Evolving Classification Function and Radial Basis Function and the two personalized methods were the Weighted k-Nearest Neighbor (WKNN) and Weighted WKNN (WWKNN) methods.

### Functional Analysis:

Metabolites and proteins that featured commonly between the first principal component of the Principal Components Analysis (PCA) and the bioinformatic methods, used above, were mapped into the Metacore network database (GeneGo, Ml, USA). Genes known to interact with or affected by the key metabolites from comparative toxicogenomics studies were also included in the analysis.

### Results:

### Metabolomics

The CS dataset has a total of 63 samples which contain 32 patient samples taken before coronary angioplasty and 31 patient samples taken approximately twenty minutes after. The CS dataset had a total of 38 attributes. 42 percent of the variance in the results obtained was accounted for by 31 metabolites identified by PCA21 of these metabolites were in the first PCA and were cross-referenced with the metabolites identified by the highest signal to noise ratio in the coronary sinus plasma dataset. 12 common metabolites were identified through this method. These metabolites were used for the functional analysis.

Discriminatory analysis using bioinformatics was applied to systemic blood taken from the aorta. Three methods were used, including wKNN classification model, a SVM classification method and an evolving spiking neural network classification model (eSNN). After 1,000 iterations of the algorithm a discriminatory accuracy of 81 percent was made. 11 features were identified using these models, five were common with the metabolites identified by PCA1 and SNR from the coronary sinus dataset.

### Proteomics

The initial analysis looking at changes within patients identified 31 proteins as significantly different between pre- and post-PCI samples (p<0.05), a further 11 were trending towards significance (p<0.10) and 23 of the proteins changed with p<0.10 also exhibited absolute consistency, i.e. all comparisons (maximum eight per proteins) showed changes in the same direction. A second analysis of the 42 proteins with p<0.10 incorporated a certain degree of individual variability by comparing post-PCI samples to pre-PCI samples from a different patient analyzed within the same run. Here, 27 proteins were identified as significantly different (p<0.05) and a further four were trending towards significance (p<0.10). This suggests that seven of the proteins were highly variable at baseline. Of the 31 proteins with p<0.10 in the second analysis, 9 were found to be consistently decreased and five consistently increased in response to PCI.

Combining the information from the two analyzes highlighted 13 proteins exhibiting absolute consistency and which changed with p<0.10 in both analyzes. Of these, four decreased by PCI had calculated ratios (post-PCI Vs pre-PCI, paired and crossed) consistently lower than <0.6, and a further three proteins were increased with all ratios >1.4.

Prior to normalization, the iTRAQ label associated with peptides with missed tryptic cleavages sites was substantially higher in the post-PCI samples compared to the corresponding pre-PCI samples (133%±10%, p=0.01). This could be related to the 1.7-fold higher levels of the endogenous trypsin inhibitor bikunin alpha microglobulin/bikunin in the post-PCI samples (p<0.05 in the paired comparison, n=8).

### Discussion:

A number of metabolites, including oleic acid and myristic acid were identified that had reasonable discriminatory value in distinguishing samples taken before and after cardiac ischemia. Although these metabolites were not organ specific only a small number of them were required to provide discriminatory confidence of 81 percent. This finding was strengthened by the fact that the same metabolites were seen not only in blood exiting the heart in the coronary sinus but were also seen in systemic blood in the aorta. This finding therefore shows a general consistency in not only patient response but also analytical methodology (from sample collection extraction through to detection and quantitation). Furthermore, the metabolites identified, when merged with proteomic data demonstrated pathway and disease mechanisms consistent with the insult being examined cardiac ischemia.

The most interesting of the metabolites identified was myristic acid (but also oleic acid) which feature strongly in the coronary sinus and aortic dataset during the signal to noise ranking, PCA and SVM processing This indicates that myristic acid can be used as a key diagnostic marker of a cardiac ischemic event, particularly in the critical period shortly after the event occurs.

The inventor(s) also believe that oleic acid and other related fatty acids may also be used in a similar diagnostic manner.

Other metabolites identified in the study included: tryptophan, glycine, lysine, isoleucine, leucine, hydroxybutyric acid, phenylalanine, valine, creatinine, threonine, aspartic acid, glutamic acid, pyroglutamic acid, alanine, cysteine, and lactic acid. The inventors also believe that these metabolites may be analyzed in combination with oleic acid, myristic acid and/or CD44 to improve the accuracy of a diagnostic method of the invention.

The inventor(s) note that myristic acid, and other fatty acids, can be detected in the breath. Accordingly, they believe breath samples may be used to detect fatty acid markers associated with cardiac ischemia and diagnose cardiac ischemia. This may have a number of advantages, allowing for quick and non-invasive diagnosis.

The proteomic analysis identified, for the first time, that CD44 (510 percent of pre-PCI samples, p<0.05, n+4) is a key marker of cardiac ischemia.

The invention has been described herein, with reference to certain preferred embodiments, in order to enable the reader to practice the invention without undue experimentation. However, a person having ordinary skill in the art will readily recognize that many of the components and parameters may be varied or modified to a certain extent or substituted for known equivalents without departing from the scope of the invention. It should be appreciated that such modifications and equivalents are herein incorporated as if individually set forth. The invention also includes all of the steps, features, compositions and compounds referred to or indicated in this specification, individually or collectively, and any and all combinations of any two or more of said steps or features.

Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in any country in the world.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1-8. (canceled)

9. A method for treating a PPAR-related disorder in a subject in need thereof, the method comprising:
administering to said subject a pharmaceutical composition comprising:
a) a micelle comprising amphiphilic co-polymers and cell targeting molecules that bind to CD44,
b) quantum dots, and
c) a medicament that treats cardiac ischemia, and
wherein said micelle contains said medicament and said quantum dots; and
wherein said PPAR-related disorder is cardiac ischemia.

10-12. (canceled)

13. The method of claim 9, wherein said cell targeting molecule is hyaluronic acid.

14. The method of claim 9, wherein said quantum dots comprise silicon.
